(19) United States
(12) Patent Application Publication
(10) Pub. No.: US 2007/0185115 A1
(43) Pub. Date: Aug. 9, 2007

(54) Novel ureido-and amido-pyrazolone derivatives

(75) Inventors: Eric Lattmann, Birmingham (GB); Pornthip Lattmann, Birminghamg (GB); Harjit Singh, Dudley (GB)

Correspondence Address: SYNNESTVEDT LECHNER & WOODBRIDGE LLP, P O BOX 592, 112 NASSAU STREET, PRINCETON, NJ, 08542-0592, US

(73) Assignee: Aston University, Birmingham (GB)

(21) Appl. No.: 10/558,787

(22) PCT Filed: May 27, 2004
(86) PCT No.: PCT/GB04/02244

§ 371 Date: Oct. 23, 2006

(30) Foreign Application Priority Data
May 30, 2003 (GB) 0312368.4

(51) Int. Cl. A61K 31/4152; C07D 237/02; C07D 403/12; A61K 31/416

(52) U.S. Cl. 514/247; 544/238; 544/239; 514/404; 548/364.1; 548/366.1

(57) ABSTRACT

The present invention provides compounds of formula (I); wherein each of R₁ to R₄ is independently selected from hydrogen, a halogen, a substituted or unsubstituted cyclic and heterocyclic moiety, substituted or unsubstituted, linear or branched alkyl, alkyloxy, alkylcarbonyl, alkyloxycarbonyl, alkenyl, alkenyloxy, alkenylcarbonyl, alkenyloxycarbonyl, alkynyl, alkynyloxy, alkynylcarbonyl, alkynyloxycarbonyl, aryl, benzyl, arlyoxy, arylcarbonyl, aryloxycarbonyl and sulphur equivalents of said oxy, carbonyl and oxycarbonyl moieties, and A is NH, or (CH₂)ₙ, where n is preferably 0, 1 or 2. The invention also relates to methods for preparing the compounds and their uses as CCK receptor ligands and CCK antagonists.

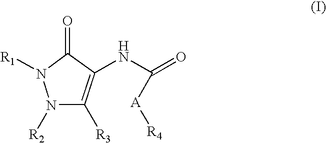

The present invention relates to novel ureido- and amido-pyrazoline derivatives, their preparation and their use as non-peptide CCK ligands, particularly in pharmaceutical formulations thereof.

Cholecystokinins (CCKs) act as anti-opioid peptides. CCK was initially described as a regulatory hormone found in endocrine cells of the gastro-intestinal (GI) tract. Some CCKs share a common amino acid sequence with gastrin, which is involved in control of gastric acid and pepsin secretion. CCKs have also been found throughout the central nervous system (CNS), where they are believed to act as a neurotransmitter and/or modulator of many important functions. There are various known structures of CCK, identified with reference to the number of amino acids they comprise. For example, CCK-8 is a naturally-occurring predominating CCK peptide and, having only eight amino acids, is the minimum fully-active sequence, although small amounts of CCK-4 may also be present.

CCK plays an important role in the invasiveness and the production of matrix metalloproteinase-9 (MMP-9) in human pancreatic cancer cell lines. The pathway of the invasiveness may be associated with MMP-9 of those lines regulated by CCK.

The gut hormone cholecystokinin exerts various actions on the gastrointestinal tract, including the regulation of growth. The hormone has been reported to induce hypertrophy and hyperplasia of the pancreas and to enhance chemically-induced pancreatic carcinogenesis in animals. Stimulation of endogenous cholecystokinin secretion through the induction of deficiency of intraintestinal proteases and bile salts by trypsin-inhibiting nutrients, bile salt-binding drugs or surgical intervention is also capable of stimulating growth and tumour development in the rat. In man, factors suggested to increase the risk of pancreatic cancer, such as a high-fat and high-protein diet or gastrectomy, are known to stimulate plasma cholecystokinin secretion. Receptors for cholecystokinin have been demonstrated on human pancreatic adenocarcinomas, and cholecystokinin has been demonstrated to enhance the growth of xenografted pancreatic cancer and to inhibit growth of gastric and bile duct cancer.

There are two subtypes of CCK receptor which were initially termed as type-A and type-B, reflecting their preferential localisation in the alimentary tract and in the brain, respectively. Recently, these receptors have been re-named as CCK1 and CCK2, respectively, although the original designation is used hereinbelow with respect to the present invention. The molecular cloning of two CCK receptor subtypes, one from rat and human pancreas and one from human brain, has confirmed the pharmacological classification of CCK receptors. Both CCK1 and CCK2 receptors belong to the family of G-protein coupled receptors. However, the differential distribution of CCK1 and CCK2 receptors in the peripheral vs. central nervous system is not absolute, and CCK1 receptors have been shown to be present in discrete regions of the CNS, including the spinal cord, particularly in primates.

The functions of the CCK1 receptors in the brain is poorly understood, whereas the CCK2 receptor is known to mediate anxiety, panic attacks, satiety and pain. Therefore, antagonists to CCK and to gastrin have been useful for preventing and treating CCK-related and/or gastrin-related disorders of the GI and CNS of animals, especially of humans. Just as there is some overlap in the biological activities of CCK and gastrin, antagonists also tend to have affinity for both receptors. In a practical sense, however, there is enough selectivity for the respective receptors that greater activity against specific CCK- or gastrin-related disorders can often also be identified.

Selective CCK antagonists are themselves useful in treating CCK-related disorders of the appetite regulatory systems of animals as well as in potentiating and prolonging opiate-mediated analgesia, thus having utility in the treatment of pain, while selective gastrin antagonists are useful in the modulation of CNS behaviour, as a palliative for gastrointestinal neoplasms, and in the treatment and prevention of gastrin-related disorders of the GI system in humans and animals, such as peptic ulcers, Zollinger-Ellison syndrome, antral G cell hyperplasia and other conditions in which reduced gastrin activity is of therapeutic value. Also, since CCK and gastrin also have trophic effects on certain tumours, antagonists of CCK and gastrin are useful in treating these tumours.

Various chemical classes of CCK-receptor antagonists have been reported. These include pyrazolidinones showing good selectivity for CCK_{B} receptors (Howbert, J. J. et. al.; Diphenylpyrazolidinone and benzodiazepine cholecystokinin antagonists: A case of convergent evolution in medicinal chemistry., Bioorg. Med. Chem. Lett. 1993, 3, 875-880.), ureidoacetamides which are potent and selective ligands for CCK_{B}/gastrin receptors (WO 91/113874), ureidophenoxyacetanilides (Takeda, Y. et. al.; Synthesis of phenoxyacetic acid derivatives as highly potent antagonists of gastrin/cholecystokinin-B receptors, Chem. Pharm Bull. 1998, 46, 951-961), ureidomethylcarbamoylphenylketones (Hagishita, S.; et. al., Ureido-methylcarbamoyl-phenylketones as selective CCK_{B} receptor antagonists. Bioorg. Med. Chem. 1997, 5, 1695-1714), and ureidobenzodiazepine derivatives (Evans, B. E.; et. al., Design of potent, orally effective, non peptidal antagonists of the peptide hormone cholecystokinin, Proc. Natl. Acad Sci. USA 1986, 83, 4918-4922).

It is an object of the present invention to provide novel ureido- and amido-pyrazoline derivatives, which preferably act as CCK ligands, and pharmaceutical formulations thereof.

According to a first aspect of the present invention, there is provided a compound of formula (1): 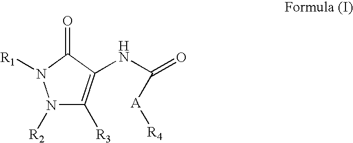
wherein

each of R₁ to R4 is independently selected from hydrogen, a halogen, a substituted or unsubstituted cyclic and heterocyclic moiety, substituted or unsubstituted, linear or branched alkyl, alkyloxy, alkylcarbonyl, alkyloxycarbonyl, alkenyl, alkenyloxy, alkenylcarbonyl, alkenyloxycarbonyl, alkynyl, alkynyloxy, alkynylcarbonyl, alkynyloxycarbonyl, aryl, benzyl, arlyoxy, arylcarbonyl, aryloxycarbonyl and sulphur equivalents of said oxy, carbonyl and oxycarbonyl moieties, and A is NH, or (CH₂)ₙ, where n is preferably 0, 1 or 2.

The scope of the invention also extends to salts, particularly physiologically acceptable salts and hydrates of the compounds of formula (I).

Preferably said alkyl-containing moieties (e.g. alkyl, alkyloxy etc.) are C₁-C₁₂, more preferably,C₁-C₆ and most preferably C₁ to C₄.

Preferably said alkenyl- and said alkynyl-containing moieties are C₂-C₁₂, more preferably C₂-C₆ and most preferably C₂ to C₄.

Preferably, said aryl moiety is substituted or unsubstituted phenyl, napthyl or indolyl. Particularly preferred are m-substituted phenyl, indol-2yl and indol-3-yl.

Examples of suitable substituents for said heterocyclic, alkyl, alkenyl, alkynyl and aryl moieties include halo, amino, nitro, hydroxy, alkoxy (eg. methoxy) and cyano moieties.

Preferably, said heterocyclic moiety is a monocyclic or bicyclic ring comprising at least one of oxygen, sulphur and nitrogen. Preferably each ring of the heterocyclic moiety is a 3 to 7 membered ring.

Preferably, said cyclic alkyl moiety is a 3 to 7 membered ring and said cyclic alkenyl and alkynyl moieties are preferably, 4 to 7 membered rings. Particularly preferred is cyclohexyl.

Preferably, R₁ is selected from H, C₁₋₄ alkyl, phenyl, benzyl, cyclohexyl, and a heterocyclic moiety. Most preferably, R₁ is phenyl.

Preferably, R₂ is selected from H, C₁₋₄ alkyl, phenyl, aryl, CH₂-heterocyclic moiety, CH₂CO-alkyl, CH₂CO-aryl, benzyl, cyclohexyl, and cycloalkyl. Most preferably, R₂ is phenyl or methyl.

Preferably, R₃ is selected from H, methyl, alkyloxy, aryloxy and a halogen (chloro and bromo derivatives being preferred). Most preferably, R₃ is methyl.

Preferably, R4 is selected from aryl, a cyclic alkyl moiety or a heterocyclic moiety. More preferably, R4 is selected from indolyl (preferably indol-2-yl) and cyclohexyl.

In those embodiments where A is NH (i.e. ureido-pyrazoline derivatives), R4 is preferably mono-substituted phenyl, t-butyl, cyclohexyl or indol-2-yl.

In those embodiments where A is (CH₂)ₙ (i.e. amido-pyrazoline derivatives), R₄ is preferably indol-2-yl or indol-3-yl.

Particularly prefered compounds in accordance with the present invention are in accordance with formulae (II) to (XV):

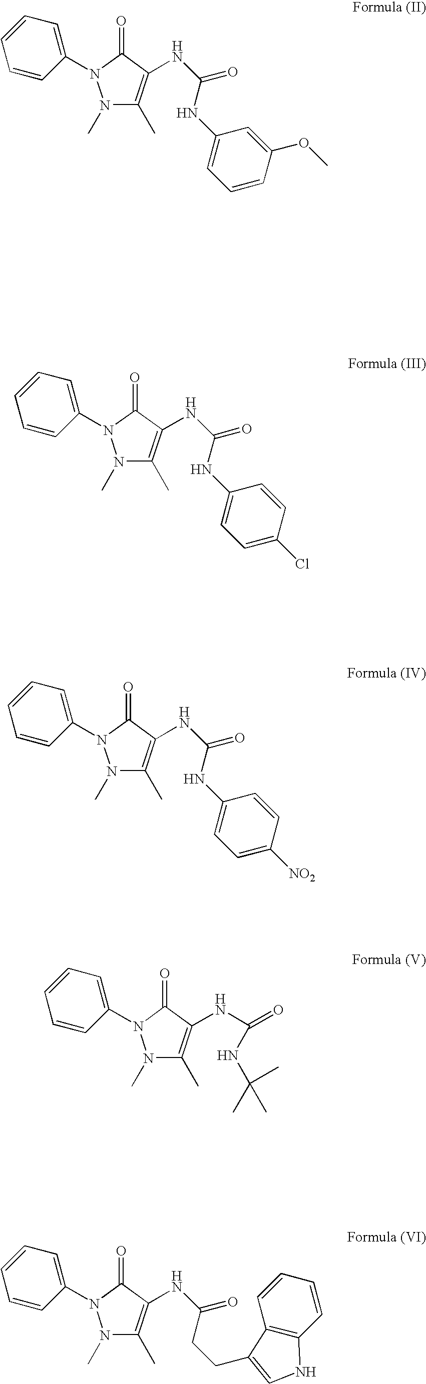

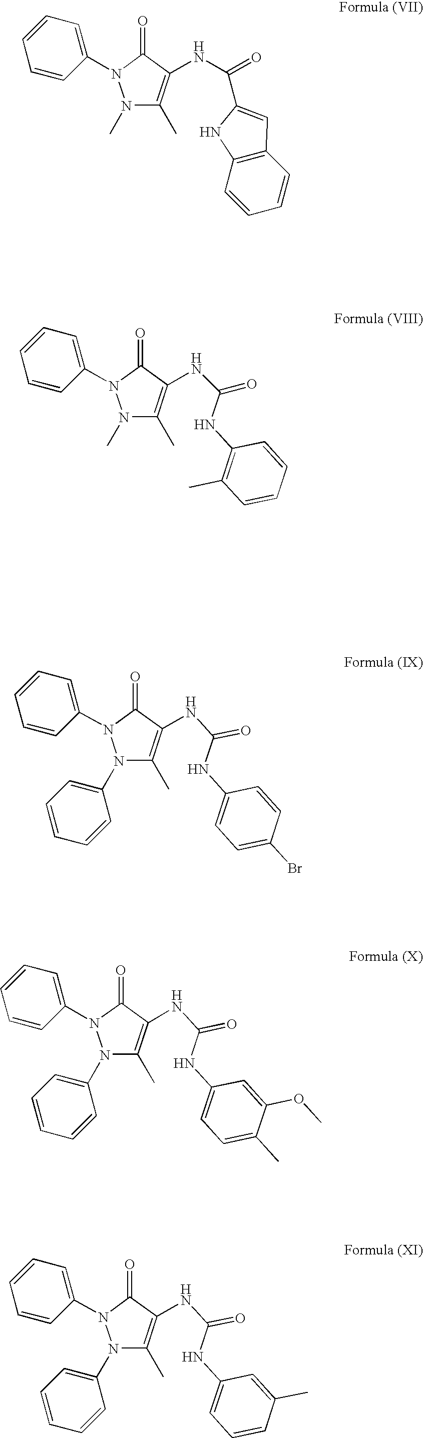

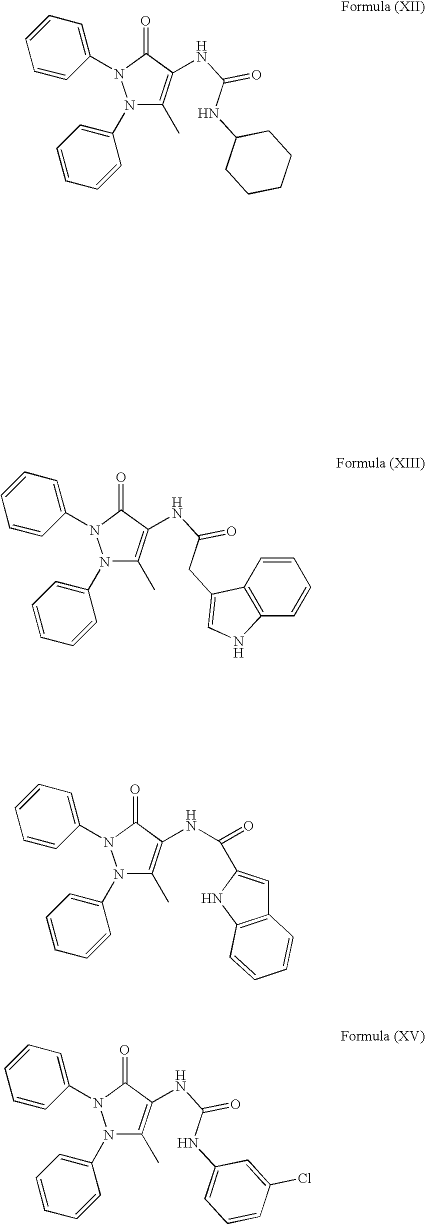

It will be understood that formula (I) is intended to embrace all possible isomers, including optical isomers and mixtures thereof, including racemates. In addition, the present invention includes within its scope prodrugs of the compounds of formula (I). In general, such prodrugs will be functional derivatives of the compounds of formula (I) which are readily convertible in vivo into the required compound of formula (I). Conventional procedures for the selection and preparation of suitable prodrug derivatives are described, for example, in “Design of Prodrugs”, ed H. Bungaard, Elsevier, 1985.

The pharmaceutically acceptable salts of the compounds of formula (I) include the conventional non-toxic salts or the quarternary ammonium salts of the compounds of formula (I) formed, eg, from non-toxic inorganic or organic acids. The pharmaceutically acceptable salts of formula (I) also include those formed from a base, such as an alkali or alkaline earth metal hydroxide, or an organic base, such as an amine or a quarternary ammonium hydroxide.

According to a second aspect of the present invention, there is provided a method of producing a compound of formula (I), comprising the sequential steps of:
(i) reacting a di-substituted hydrazine derivative of formula (1) with a β-ketoester of formula (2) to produce a pyrazolone of formula (3),
(ii) introducing an amine group at the 4-position of the pyrazolone (3) to produce 4-aminopyrazolone (4), and
(iii) reacting the aminopyrazolone (4) with either an isocyanate of formula (5) to produce the desired ureido-pyrazolone (6), or a carboxylic acid of formula (7) to produce the desired amido-pyrazolone (8).

The method of the second aspect is illustrated in scheme 1 below in which R₁ to R4 and n are as previously defined.

In cases where R₂ is H, the method may include the additional step of alkylating the pyrazolone (3) prior to step (ii).

The di-substituted hydrazine used in step (i) is conveniently a phenylhydrazine (R₁=Ph).

Step (ii) is conveniently achieved by introducing a nitroso group at the 4position of the pyrazolone (3) followed by reduction.

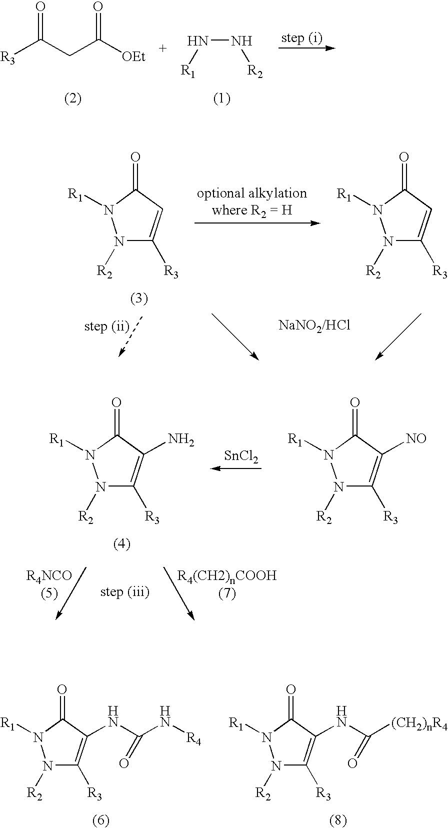

The skilled person will readily be able to determine optimum reagents and conditions for carrying out the steps of the method, however, the following is given as an illustrative example. The pyrazol-3-one ring structure can be built up from ethyl acetoacetate (R₃=CH₃) and phenylhydrazine at a temperature of about 180-200° C. in the absence of a solvent. Alkylation at N−1 is then conveniently achieved by deprotonation with base, such as by forming a suspension with NaH, in mineral oil, under inert conditions, followed by addition of an appropriate alkylating agent eg benzylbromide (R₃=Bn) or chloropinacolone (R₃=^{t}BuC(O)CH₂) under mild conditions, such as at room temperature.

Nitrosation at the 4-position can be achieved using standard methods, such as by reaction with sodium nitrite in the presence of concentrated, aqueous mineral acid eg hydrochloric acid, at reduced temperature, such as at 0° C. Reduction with a suitable reducing agent, such as tin chloride, gives the 4-amino derivative and a tin hydroxide by-product.

The product is then dissolved in acetonitrile, treated with an isocyanate (eg. phenylisocyanate, R₄=Ph) and heated to about 50 to 70° C., the tin hydroxide remaining undissolved. Filtration and removal of the acetonitrile in vacuo provides the crystalline ureido-pyrazolone product. Alternatively, the 4-amino derivative can be reacted with a carboxylic acid (eg. indole-2-carboxylic acid, R₄=indol-2-yl, A=(CH₂)ₙ, n=0)) in the presence of DIC , preferably at elevated temperature in the range of 50-70° C., to produce the amido-pyrazolone product.

The present invention also resides in the use of a compound of the first aspect as a CCK receptor-ligand and/or as a CCK antagonist. Preferably, said use is as a selective CCK1 or CCK2 ligand.

The abiiity of the compounds of formula (I) to antagonise CCK by acting as CCK-receptor ligands makes these compounds useful as pharmacological agents for mammals, especially humans, for the treatment and prevention of disorders wherein CCK and/or gastrin may be involved.

Therefore the present invention in a third aspect resides in a method of treatment of a mammal afflicted with a CCK-related condition, or prophylaxis in a mammal at risk of a CCK-related condition by administration of a therapeutically effective amount of a compound of the first aspect of the invention.

The invention also resides in a-pharmaceutical formulation comprising a compound of said first aspect in admixture with a pharmaceutically acceptable carrier therefor.

The invention fturther resides in the use of a compound of the first aspect in the preparation of a medicament, particularly a medicament for the treatment or prophylaxis of a CCK-related disorder.

Examples of CCK-related conditions states include GI disorders, especially such as irritable bowel syndrome, gastro-oesophageal reflux disease or ulcers, excess pancreatic or gastric secretion, acute pancreitis, or motility disorders; CNS disorders caused by CCK interactions with dopamine, such as neuroleptic disorders, tardive dyskinesia, Parkinson's disease, psychosis or Gilles de la Tourette syndrome; disorders of appetite regulatory systems; Zollinger-Ellison syndrome; antral G cell hyperplasia; or pain (potentiation of opiate analgesia).

The treatment of opiate-resistant severe clinical pain may represent the most important of the CNS applications, but other applications based on the interaction between CCK and dopamine in forebrain could also deserve clinical exploration

The compounds of the invention may further be useful in the treatment or prevention of additional central nervous system disorders including neurological and psychiatric disorders. Example of such central nervous system disorders include anxiety disorders and panic disorders, wherein CCK is involved. Additional examples of central nervous system disorders include panic syndrome, anticipatory anxiety, phobic anxiety, panic anxiety, chronic anxiety and endogeneous anxiety.

The compounds of of the invention may further be useful in the treatment of oncologic disorders wherein CCK may be involved. Examples of such oncologic disorders include small cell adenocarcinomas and primary tumours of the central nervous system glial and neuronal cells. Example of such adenocarcinomas and tumours include, but are not limited to, tumours of the lower oesophagus, stomach, intestine, colon and lung, including small cell lung carcinoma

The compounds of the invention may further be used to control pupil constriction in the eye. The compounds may be used for therapeutic purposes during eye examinations and intra-ocular surgery in order to prevent miosis. They may further be used to inhibit miosis occurring in association with iritis, uveitis and trauma.

The compounds of the invention may further be useful for preventing or treating the withdrawal response produced by chronic treatment-or abuse of drugs or alcohol. Such drugs include, but are not limited to, cocaine, alcohol or nicotine.

The compounds of the invention may also be useful as neuroprotective agents, for example, in the treatment and/or prevention of neuro-degenerative disorders arising as consequence of such pathological conditions as stroke, hypoglycaemia, cerebral palsy, transient cerebral ischaemic attack, cerebral ischaemia during cardiac pulmonary surgery or cardiac arrest, perinatal asphyxia, epilepsy, Huntingdon's chorea, Alzheimer's disease, amyotrophic lateral sclerosis, Parkinson's disease, olivo-pontocerebellar atrophy, anoxia such as from drowning, spinal cord and head injury, and poisoning by neurotoxins, including environmental neurotoxins.

The dosage administered to a patient will normally be determined by the prescribing physician and will generally vary according to the age, weight and response of the individual patient, as well as the severity of the patient's symptoms. However, in most instances, an effective therapeutic daily dosage will be in the range of from about 0.05 mg/kg to about 50 mg/kg of body weight and, preferably, of from 0.5 mg/kg to about 20 mg/kg of body weight administered in single or divided doses. In some cases, however, it may be necessary to use dosages outside these limits.

In the treatment of irritable bowel syndrome, for instance, 0.1 to 10 mg/kg of a CCK antagonist might be administered orally (p.o.), divided into two doses per day (b.i.d.). In treating delayed gastric emptying, the dosage range would probably be the same, although the drug might be administered either intravenously (i.v.) or orally, with the i.v. dose probably tending to be slightly lower due to a better availability. Acute pancreitis might be treated preferentially in an i.v. form, whereas spasm and/or reflex oesophageal, chronic pancreitis, post-vagotomy diarrhoea, anorexia or pain associated with biliary dyskinesia might indicate a p.o. form of administration.

In the effective treatment of panic syndrome, panic disorder, anxiety disorder and the like, preferably about 0.05 mg/kg to about 1.0 mg/kg of CCK antagonist may be administered orally (p.o.), in single or divided doses per day (b.i.d.). Other routes of administration are also suitable.

For directly inducing analgesia, anaesthesia or loss of pain sensation, the effective dosage range is preferably from about 100 mg/kg to about 1 mg/kg by intraperitoneal administration. Oral administration is an altemative route, as well as others.

While it is possible for an active ingredient to be administered alone as the raw chemical, it is preferable to present it as a pharmaceutical formulation. The formulations, both for veterinary and for human medical use, of the present invention comprise an active ingredient in association with a pharmaceutically acceptable carrier therefor and optionally other therapeutic ingredient(s). The carrier(s) must be ‘acceptable’ in the sense of being compatible with the other ingredients of the formulation and not deleterious to the recipient thereof.

Conveniently, unit doses of a formulation contain between 0.1 mg and 1 g of the active ingredient. Preferably, the formulation is suitable for administration from one to six, such as two to four, times per day. For topical administration, the active ingredient preferably comprises from 1% to 2% by weight of the formulation but the active ingredient may comprise as much as 10% w/w. Formulations suitable for nasal or buccal administration, such as the self-propelling powder-dispensing formulations described hereinafter, may comprise 0.1 to 20% w/w, for example about 2% w/w of active ingredient.

The formulations include those in a form suitable for oral, ophthalmic, rectal, parenteral (including subcutaneous, vaginal, intraperitoneal, intramuscular and intravenous), intra-articular, topical, nasal or buccal administration.

Formulations of the present invention suitable for oral administration may be in the form of discrete units such as capsules, cachets, tablets or lozenges, each containing a predetermined amount of the active ingredient; in the form of a powder or granules; in the form of a solution or a suspension in an aqueous liquid or non-aqueous liquid; or in the form of an oil-in-water emulsion or a water-in-oil emulsion. The active ingredient may also be in the form of a bolus, electuary or paste. For such formulations, a range of dilutions of the active ingredient in the vehicle is suitable, such as from 1% to 99%, preferably 5% to 50% and more preferably 10% to 25% dilution. Depending upon the level of dilution, the formulation will be either a liquid at room temperature (in the region of about 20° C.) or a low-melting solid.

Formulations for rectal administration may be in the form of a suppository incorporating the active ingredient and a carrier such as cocoa butter, or in the form of an enema.

Formulations suitable for parenteral administration comprise a solution, suspension or emulsion, as described above, conveniently a sterile aqueous preparation of the active ingredient that is preferably isotonic with the blood of the recipient.

Formulations suitable for intra-articular administration may be in the form of a sterile aqueous preparation of the active ingredient, which may be in a microcrystalline form, for example, in the form of an aqueous microcrystalline suspension or as a micellar dispersion or suspension. Liposomal formulations or biodegradable polymer systems may also be used to present the active ingredient particularly for both intra-articular and ophthalmic administration.

Formulations suitable for topical administration include liquid or semi-liquid preparations such as liniments, lotions or applications; oil-in-water or water-in-oil emulsions such as creams, ointments or pastes; or solutions or suspensions such as drops. For example, for ophthalmic administration, the active ingredient may be presented in the form of aqueous eye drops, as for example, a 0.1-1.0% solution.

Drops according to the present invention may comprise sterile aqueous or oily solutions. Preservatives, bactericidal and fungicidal agents suitable for inclusion in the drops are phenylmercuric salts (0.002%), benzalkonium chloride (0.01%) and chlorhexidine acetate (0.01%). Suitable solvents for the preparation of an oily solution include glycerol, diluted alcohol and propylene glycol.

Lotions according to the present invention include those suitable for application to the eye. An eye lotion may comprise a sterile aqueous solution optionally containing a bactericide or preservative prepared by methods similar to those for the preparation of drops. Lotions or liniments for application to the skin may also include an agent to hasten drying and to cool the skin, such as an alcohol, or a softener or moisturiser such as glycerol or an oil such as castor oil or arachis oil.

Creams, ointments or pastes according to the present invention are semi-solid formulations of the active ingredient in a base for external application. The base may comprise one or more of a hard, soft or liquid paraffin, glycerol, beeswax, a metallic soap; a mucilage; an oil such as a vegetable oil, eg almond, corn, arachis, castor or olive oil; wool fat or its derivatives; or a fatty acid ester of a fatty acid together with an alcohol such as propylene glycol or macrogols. The formulation may also comprise a suitable surface-active agent, such as an anionic, cationic or non-ionic surfactant such as a glycol or polyoxyethylene derivatives thereof. Suspending agents such as natural gums may be incorporated, optionally with other inorganic materials, such as silicaceous silicas, and other ingredients such as lanolin.

Formulations suitable for administration to the nose or buccal cavity include those suitable for inhalation or insufflation, and include powder, self-propelling and spray formulations such as aerosols and atomisers. The formulations, when dispersed, preferably have a particle size in the range of 10 to 200μ.

Such formulations may be in the form of a finely comminuted powder for pulmonary administration from a powder inhalation device or self-propelling powder-dispensing formulations, where the active ingredient, as a finely comminuted powder, may comprise up to 99.9% w/w of the formulation.

Self-propelling powder-dispensing formulations preferably comprise dispersed particles of solid active ingredient, and a liquid propellant having a boiling point of below 18° C. at atmospheric pressure. Generally, the propellant constitutes 50 to 99.9% w/w of the formulation whilst the active ingredient constitutes 0.1 to 20% w/w. for example, about 2% w/w, of the formulation.

The pharmaceutically acceptable carrier in such self-propelling formulations may include other constituents in addition to the propellant, in particular a surfactant or a solid diluent or both. Surfactants are desirable since they prevent agglomeration of the particles of active ingredient and maintain the active ingredient in suspension. Especially valuable are liquid non-ionic surfactants and solid anionic surfactants or mixtures thereof. Suitable liquid non-ionic surfactants are those having a hydrophile-lipophile balance (HLB, see Journal of the Society of Cosmetic Chemists Vol. 1 pp. 311-326 (1949)) of below 10, in particular esters and partial esters of fatty acids with aliphatic polyhydric alcohols. The liquid non-ionic surfactant may constitute from 0.01 up to 20% w/w of the formulation, though preferably it constitutes below 1% w/w of the formulation. Suitable solid anionic surfactants include alkali metal, ammonium and amine salts of dialkyl sulphosuccinate and alkyl benzene sulphonic acid. The solid anionic surfactants may constitute from 0.01 up to 20% w/w of the formulation, though preferably below 1% w/w of the composition. Solid diluents may be advantageously incorporated in such self-propelling formulations where the density of the active ingredient differs substantially from the density of the propellant; also, they help to maintain the active ingredient in suspension. The solid diluent is in the form of a fine powder, preferably having a particle size of the same order as that of the particles of the active ingredient. Suitable solid diluents include sodium chloride, sodium sulphate and sugars.

Formulations of the present invention may also be in the form of a self-propelling formulation wherein the active ingredient is present in solution. Such self-propelling formulations may comprise the active ingredient, propellant and co-solvent, and advantageously an antioxidant stabiliser. Suitable co-solvents are lower alkyl alcohols and mixtures thereof. The co-solvent. may constitute 5 to 40% w/w of the formulation, though preferably less than 20% w/w of the formulation. Aitioxidant stabilisers may be incorporated in such solution-formulations to inhibit deterioration of the active ingredient and are conveniently alkali metal ascorbates or bisulphites. They are preferably present in an amount of up to 0.25% w/w of the formulation.

Formulations of the present invention may also be in the form of an aqueous or dilute alcoholic solution, optionally a sterile solution, of the active ingredient for use in a nebuliser or atomiser, wherein an accelerated air stream is used to produce a fine mist consisting of small droplets of the solution. Such formulations usually contain a flavouring agent such as saccharin sodium and a volatile oil. A buffering agent such as sodium metabisulphite and a surface-active agent may also be included in such a formulation which should also contain a preservative such as methylhydroxybenzoate.

Other formulations suitable for nasal administration include a powder, having a particle size of 20 to 500 microns, which is administered in the manner in which snuff is taken, ie by rapid inhalation through the nasal passage from a container of the powder held close up to the nose.

In addition to the aforementioned ingredients, the formulations of this invention may include one or more additional ingredients such as diluents, buffers, flavouring agents, binders, surface active agents, thickeners, lubricants, preservatives eg methylhydroxybenzoate (including anti-oxidants), emulsifying agents and the like. A particularly preferred carrier or diluent for use in the formulations of this invention is a lower alkyl ester of a C₁₈ to C₂₄ mono-unsaturated fatty acid, such as oleic acid, for example ethyl oleate. Other suitable carriers or diluents include capric or caprylic esters or triglycerides, or mixtures thereof, such as those caprylic/capric triglycerides sold under the trade name Miglyol, eg Miglyol 810.

Because these compounds antagonise the function of CCK in animals, they may also be used as feed additives to increase the food intake of animals, such as in a daily dosage of from about 0.05 to 50 mg/kg of body weight.

The present invention will now be exemplified with reference to the following Examples

### EXAMPLES

### Preparation of Intermediates & Starting Materials

### Description 1:

### Preparation of 5Methyl-2-phenyl-1,2-dihdyro-3H-pyrazol-3-one

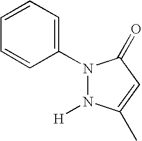

Phenyl hydrazine (15.0 g, 0.14 mol, 1 Eq.) was added slowly to acetic acid ester (2 Eq, 36.0 ml, 0.28 mol) at 180° C. in neat condition. The mixture was allowed to heat over 3 hours and then cooled to room temperature. The mixture was washed with ethanol to remove an excess of unreacted starting materials. Then, it was filtered to give a white precipitate, which was subsequently recrystallised from ethanol.

Yield: 71%. Mol. Weight: 174.2 Mol. Formula: C₁₀H₁₀N₂O. MS (APCI(+)): 175 (M+1) m/z. IR (KBr-disc) ν max: 3438, 3063, 2685, 1592, 1557, 1492, 1455, 1407, 1293, 750 & 703 cm⁻¹. ¹H NMR (DMSO-d₆) 300K δ: 2.11 (s, CH₃), 5.62 (s, CH), 7.37-7.43 (t, Ar-2H, J=7.6, 7.9 Hz), 7.53-7.59 (t, Ar—H, J=7.4, 7.5 Hz), 7.68-7.72 (d, Ar-2H, J=8.7 Hz) p.p.m.

### Description 2:

### Alkylating Method for Alkylation of Compounds where R₂=H to Compounds where R₂≠H

A suspension of 50% sodium hydride in mineral oil (0.03 mol) was added in drops to a solution of 5-methyl-2-phenyl-1,2-dihydro-3H-pyrazol-3-one (4.35 g, 0.025 mol), prepared as in Description 1, in dry DMF: (50.0 ml). After stirring for 20 mins at RT, under inert conditions, the relevant alkylating agent (0.03 mol) was added in drops to the mixture, under ice cooling. The mixture was stirred for an additional 35 mins at RT. After 35 mins, water was added and ethylacetate was added to the suspension. The organic extract was washed with brine, dried over sodium sulphate and the solvent removed in vacuo. Column chromatography afforded the pure products.

By this method, were prepared:

### Description 2a

### 1-(2,2-Diethyl propanoyl)-5-methyl-2-phenyl-1,2-dihydro-3H-pyrazol-3-one

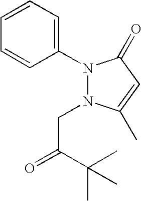

Yield: 42.5%. R_{f} (50% ether in 40-60 petroleum ether)=0.63. Mol. Weight: 258.3. Mol. Formula: C₁₅H₁₈N₂O₂. MS (APCI(+)): 259 (M+l) m/z. IR(KBr-disc)b) ν max: 3442, 2963, 1721, 1600, 1511, 1476, 1451, 1394, 1162, 1054, 918 & 762 cm⁻¹. ¹H NMR (CDCI₃) 300K δ: 0.97 (s, CH₃), 1.18 (s, (CH₃)₃), 5.01(s, CH), 7.23-7.28 (t, Ar—H, J=7.4, 7.5 Hz), 7.38-7.44 (t, Ar-2H, J=7.5, 7.8 Hz), 7.70-7.74 (d, Ar-2H, J=8.0 Hz ) p.p.m. ¹³C NMR (CDCl₃) 300K δ: 26.1 (C(CH₃)₃), 45.5 (C(CH₃)₃), 122.0 (2×C), 126.4, 128.7 (2×C), 138.3 (Ar—C), 149.1 (CH), 162.5 (C═O), 194.0 (C═O) p.p.m.

### Description 2b

### Preparation of 1-Benzyl-5-methyl-2-phenyl-1,2-dihydro-3H-pyrazol-3-one

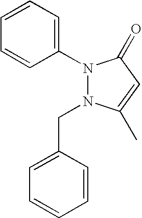

Yield: 37.1%. Rf (50% ether in 40-60 petroleum ether)=0.61. Mol. Weight: 264.3. Mol. Formula: C₁₇H₁₆N₂O. MS (APCI(−)): 263 (M−1) m/z. IR (KBr-disc) ν max: 3201, 3062, 3010, 2915, 2866, 1754, 1694, 1542, 1476, 1205 & 747 cm⁻¹. ¹H NMR (CDCl₃) 300K δ: 1.31 (s, CH₃), 4.50 (s,—CH₂—), 4.77 (s, CH), 7.12-7.56 (m, Ar-10H) p.p.m.

### Description 3

### Preparation of 5-Methyl-1,2-diphenyl-1,2-dihydro-3H-pyrazol-3-one

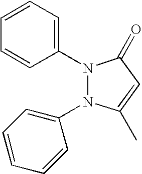

Diphenyl hydrazine (50.0 g, 0.27 mol) and acetic acid ester (2 Eq. 69.0 ml, 0.52 mol) were heated at 130-150° C. for 2 hours, with a Dean stark trap. The mixture was then heated to an additional 1.5 hours at 180° C., to remove water, ethanol and acetic acid ester. The remaining solution was distilled at 230-250° C. at 2mm Hg. This removed any unreacted diphenyl hydrazine to give a viscous black liquid. The mixture was allowed to cool to RT and then ether was added to precipitate out crude black crystals. These were subsequently recrystallised twice from toluene.

Yield: 32.8%. Mol. Weight: 250.3. Mol. Formula: C₁₆H₁₄N₂O. MS (APCI(+)): 25.1 (M+1) m/z. IR (KBr disc) ν max: 3465, 3090, 1671, 1590, 1490, 1380, 1349, 1241, 971, 753 & 688 cm⁻¹. ¹H NMR (CDCl₃) 300K δ: 2.07 (s, CH₃), 5.55 (s, CH), 7.05-7.37 (m, Ar-10H) p.p.m. ¹³C NMR (CDCl₃) 300K δ: 13.7 (CH₃), 99.2 (CH), 123.6 (2×C), 125.5 (2×C), 125.9 (2×C), 128.0, 128.6 (2×C), 129.3, 135.7, 139.0 (Ar—C), 156.3 (C—N), 166.5 (C═O) p.p.m.

### Description 4

### Preparation of 4-Nitro-5-methyl-1,2-diphenyl-1,2-dihydro-3H-pyrazol-3-one

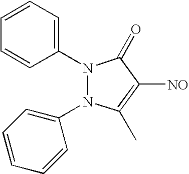

5-Methyl-1,2-diphenyl-1,2-dihydro-3H-pyrazol-3-one (10.0 g, 0.04 mol), prepared as in Description 3, was warmed in HCl (conc) (60.0 ml). When dissolved the solution was diluted with water (up to 400 ml). Sodium nitrite (2.8 g; 0.041 mol) in water (50.0 ml) was added in drops to the mixture at 0° C., whilst stirring. A green precipitate was produced, which was allowed to stand for 45 mins, then filtered, washed with cold water and dried.

### Description 5

### Preparation of 4Amino-5-methyl-1,2-diphenyl-1,2-dihydro-3H-pyrazol-3-one

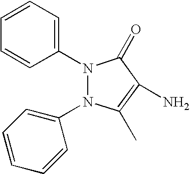

4-Nitroso-5-methyl-1,2-diphenyl-1,2-dihydro-3H-pyrazol-3-one, (8.5 g, 0.04 mol), prepared as in Description 4, was dissolved in ethanol (250 ml). A mixture of tin chloride (20.4 g, 0.11 mol) in 20% HCI (120 ml) was heated to 90° C. When dissolved, the hot mixture was added to the alcoholic solution and allowed to cool to RT, and allowed to stand overnight. Ammonia solution (conc 33%) was added to the mixture until no further precipitation occurred. The mixture was filtered, dried and extracted several times with ethanol. The ethanol was removed in vacuo and the crude mixture was recrystallised in ethanol to give bright yellow crystals.

Yield: 37.0%. Mol. Weight: 265.3. Mol. Formula: C₁₆H₁₅ N₃O. MS (APCI(+)): 266 (M+1), 251 (M+) m/z. IR(KBr-disc) ν max: 3407, 3210, 1654, 1592, 1492, 1351, 1262, 751 & 690 cm⁻¹. ¹H NMR (DMSO-d₆) 300K δ: 1.88 (s, CH₃), 5.57 (s, CH), 7.05-7.12 (tt, Ar—H, J=7.3 Hz), 7.20-7.45 (m, Ar-9H) p.p.m. ¹³C NMR (DMSO-d₆) 300K δ: 11.09 (CH₃), 120.3, 122.5 (2×C), 123.8, 125.5 (2×C), 128.0, 129.1 (2×C), 129.8 (2×C). (Ar—C), 136.4 (CH), 142.7 (Ar—C), 156.3, 166.3 (C═O) p.p.m.

### Examples 1 to 3

A solution of the relevant amine in dry acetonitrile was stirred at room temperature. The appropriate isocyanate (1-phenyl/1-napthyl, 1.1 Eq) in dry acetonitrile (20 ml) was added slowly over 5 minutes, allowed to stir at room temperature or heated to 60° C. and left overnight. The precipitate that formed was filtered, washed (twice) and dried, to give the corresponding urea product.

### Example 1

### N-(1,5-dimethyl-3-oxo-2-diphenyl-2,3-dihydro-1H-pyrazol4-yl)-N′-phenylurea

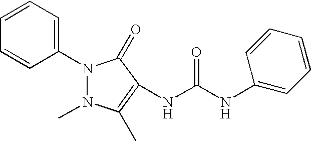

Yield: 94%. Mol. Weight: 322.4. Mol. Formula: C₁₈ H₁₈N₄O₂. MS (APCI(+)): 323 (M+1) m/z. IR (KBr-disc),ν max: 3318, 3279, 3139, 1700, 1642, 1586, 1550, 1496, 1311, 1210, 737, & 699 cm⁻¹. ¹H NMR (DMSO-d₆) 300K δ: 2.21 (s, CH₃), 3.04 (s, N—CH₃), 6.91-6.97 (t, Ar—H, J=7.3 Hz), 7.22-7.53 (m, Ar-9H), 8.80 (s, NH) p.p.m. ¹³C NMR (DMSO-d₆) 300K δ: 11.7 (CH₃), 36.6 (N—CH₃), 108.7 (CH—NH), 118.6 (2×C), 122.2, 123.9 (2×C), 126.7, 129.2 (2×C), 129.6 (2×C), 135.5 (C—CH₃), 140.2, 152.2 (Ar—C), 154.2, 162.7 (C═O) p.p.m.

### Example 2

### N-(1,5-dimethyl-3-oxo-2-diphenyl-2,3-dihydro-1H-pyrazol4-yl)-N-(1-naphthyl)urea

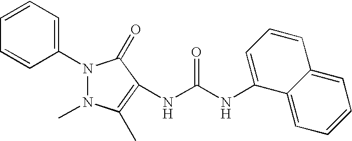

Yield: 91%. Mol. Weight: 372.4. Mol. Formula: C₂₂H₂₀N₄O₂. MS (APCI(+)): 373 (M+1) m/z. IR(KBr-disc) ν max: 3280, 3044, 1663, 1638, 1565, 1496, 1317, 1253, 780 & 668 cm⁻¹ ¹³H NMR (DMSO-d₆) 300K δ: 2.26 (s, CH₃), 3.04 (s, N—CH₃), 7.29-7.32 (t, Ar—H, J=7.2 Hz), 7.38-7.67 (m, Ar-8H) 7.93-7.99 (t, Ar—H, 7.4, 7.6 Hz), 7.99-8.02 (d, Ar—H, J=7.4 Hz); 8.14-8.17 (d, Ar—H, J=7.9 Hz), 8.95 (s, NH), 9.20 (s, NH) p.p.m.

### Example 3

### N-(4-chlorophenyl)-N′-(1,5-dihiethyl-3-oxo-2-phenyl-2,3-dihydro-1H-pyrazol-4-yl)urea

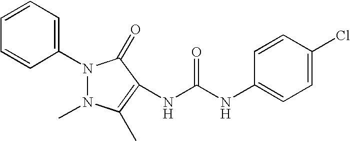

Yield: 75%. Mol. Weight: 418.9. Mol. Formula: C₂₃H₁₉ClN₄O₂. MS (APCI(+)): 419, 421 (M+1) m/z. IR (KBr-disc) ν max: 3371, 3210, 3059, 1698, 1656, 1512, 1319, 747 & 655 cm⁻¹. ¹H NMR (DMSO-d₆) 300K δ: 2.19 (s, CH₃), 3.04 (s, N—CH₃), 7.27-7.53 (m, Ar-9H), 8.95 (s, NH) p.p.m.

Examples 4 to 15 were also prepared by analogous methods.

### Examples 16 and 17

A solution of the appropriate pyrazolinone was dissolved in dry acetonitrile (20 ml). The appropriate indole acid (1.25 Eq) was added, with DIC (3 Eq). The mixture was heated to 60° C. and left overnight. The resulting precipitated crystals were filtered, washed and dried.

### Example 16

### N-(1,5-dimethyl-3-oxo-2-diphenyl-2,3-dihydro-1H-pyrazol4yl)-1H-indole-3-carboxamide

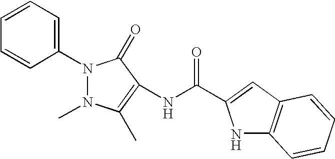

Yield: 76%. Mol. Weight: 346.4. Mol. Formula: C₂₀H₁₈N₄O₂. MS (APCI(+)): 347 (M+1), 329 (M+) m/z. IR (KBr-disc) ν max: 3337, 3307, 2965, 1696, 1696, 1623, 15557, 1363, 1251 & 826 cm⁻¹. ¹H NMR (DMSO-d₆) 300K δ: 2.18.(s, CH₃), 3.10 (s, N—CH₃), 5.50 (s, C═CH—), 7.02-7.06 (t, Ar—H, J=7.4 Hz), 7.18-7.22 (t, Ar—H, J=7.2, 7.3 Hz), 7.33-7.51 (m, Ar-6H), 7.62-7.65 (d, Ar—H, J=8.0 Hz), 9.51 (s, NH), 11.56 (s, NH) p.p.m.

### Example 17

### N-(1,5-Dimethyl-3-oxo-2-phenyl-2 3dihydro-1H-pyrazol-4yl)-3-(1H-indol-3-yl)propanamide

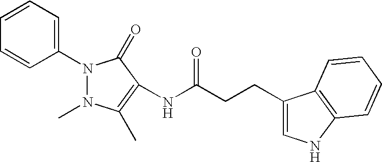

Yield: 75%. Mol. Weight: 374.4. Mol. Formula: C₂₂H₂₂N₄O₂. MS (APCI(+)): 375 (M+1) m/z. IR (KBr-disc) ν max: 3421, 3311, 3059, 2843, 1676, 1645, 1543, 1529, 1487, 1395, 1240 & 704 cm⁻¹. ¹HNMR (DMSO-d₆) 300K δ: 2.01 (s, CH₃), 2.61-2.69 (t, CH₂, J=8.1, 7.9 Hz), 3.02 (s, N—CH₃), 3.57-3.74 (m, CH₂), 6.93-7.58 (m, Ar-9H), 8.22-8.26 (d, Ar—H, J=7.8 Hz), 9.08 (s, NH), 10.76 (s, NH) p.p.m. ¹³C NMR (DMSO-d₆) 300K δ: 22.2 (CH₃), 20.8 (CH₂), 23.8 (CO—CH₂), 36.5 (N—CH₃), 108.3 (C—NH), 111.9, 118.7, 122.5, 122.8, 123.9, 126.7 (2×C), 127.5; 127.6 (2×C), 129.6, 135.6, 136.7 (Ar—C), 162.4, 170.2 (C═O) p.p.m.

Examples 18 to 20 were also prepared by analogous methods

### Examples 21 to 27

4-Amino-5-methyl-1,2-diphenyl-1,2-dihydro-3H-pyrazol-3-one (0.1 g, 3.8×10⁻⁴ mol), prepared as in Description 5, in dry acetonitrile (10-15 ml ) was stirred at room temperature. The appropriate substituted isocyanate (1.3 Eq) in dry acetonitrile was added slowly over 5 minutes, allowed to stir at room temperature or heated to 60° C. and left overnight. The precipitate that formed was filtered, washed (twice) and dried, to give the corresponding pure urea product.

### Example 21

### N-(5-methyl-3-oxo-1,2-diphenyl-2,3-dihydro-1H-pyrazol-4-yl)N′-3-methoxyphenylurea

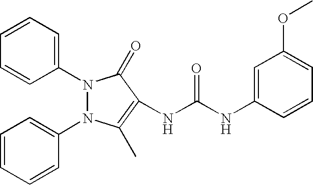

Yield: 67%. Mol. Weight: 414.6. Mol. Formula: C₂₄H₂₂N₄O₃. MS (APCI(+)): 415 (M+1), 266 (M+) m/z. IR (KBr-disc) ν max: 3207, 1708, 1646, 1619, 1594, 1540, 1488, 1453, 1282, 761 & 697 cm⁻¹. ¹H NMR (DMSO-d₆) 300K δ: 2.02n(s, C—CH₃), 3.72 (s, OCH₃), 6.50-6.55 (dd, Ar—H, J=8.2 Hz), 6.88-6.92 (Ar—H, J=8.1 Hz), 7.12-7.18 (m, Ar-3H), 7.26-7;44 (m, Ar-9H), 7.57 (s, NH), 8.88 (s, NH) p.p.m. ¹³C NMR (DMSO-d₆) 300K δ: 12.6 (C-CH₃), 55.4 (OCH₃), 99.7 (C—CH₃), 104.2, 107.7, 109.7, 110.8, 123.6 (2×C), 125.6 (2×C), 126.2, 128.6, (2×C), 130.0 (2×C), 136.1, 139.2, 141.6, 143.0, 151.2, 153.8 (Ar—C), 160.2, 163.0 (C═O) p.p.m.

### Example 22

### N-(5-Methyl-3-oxo-1,2-diphenyl-2,3-dihydro-1H-pyrazol4-yl)N′-3-methylphenylurea

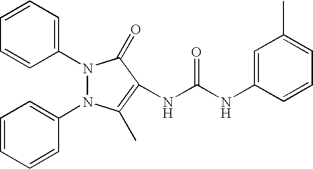

Yield: 91%. Mol. Weight: 398.5. Mol. Formula: C₂₄H₂₂N₄O₂. MS (APCI(+)): 399 (M+1), 266 (M+) m/z. IR (KBr-disc) ν max: 3322, 1698, 1644, 1625, 1538, 1490, 1285, 1211, 759 & 697 cm-^{31 1}. ¹H NMR (DMSO-d₆) 300K δ: 2.01 (s, CH₃), 2.25 (s, C—CH₃), 6.75-7.78 (d, Ar—H, J=7.2 Hz), 7.10-7.44.(m, Ar-13H), 7.59 (s, NH), 8.80 (NH) p.p.m. ¹³C NMR (DMSO-d₆) 300K δ: 12.7 (C—CH₃), 21.7 (CH₃), 109.9, 115.7, 119.1, 123.0, 123.7 (2×C), 126.4, 128.7, 129.1 (2×C), 130.1 (2×C), 136.1, 138.4, 139.9, 140.2, 142.9, 151.1 (Ar—C), 153.9, 163.0 (C═O) p.p.m.

### Example 23

### N-(2-Chlorophenyl)-N′-(5methyl-3-oxo-1,2-diphenyl-2,3-dihydro-1H-pyrazol-4-yl)urea

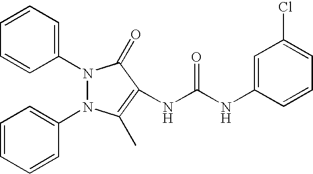

Yield: 73%. Mol. Weight: 418.9. Mol. Formula: C₂₃H₁₉N₄O₂. MS (APCI(+)): 418, 420 (M+1), 266 (M+) m/z. IR (KBr-disc) ν max: 3293, 3212, 1710, 1621, 1590, 1530, 1488, 1422, 1291, 1191, 761 & 680 cm⁻¹. ¹HNMR (DMSO-d₆)300K δ: 2.01 (s, CH₃), 6.97-7.03 (tt, Ar—H, J=6.8 Hz), 7.11-7.18 (tt, Ar—H, J=6.9, 6.8 Hz), 7.22-7.44 (m, Ar-12H), 7.89 (s, NH), 9.09 (s, NH) p.p.m. ¹³C NMR (DMSO-d₆) 300K δ: 12.6 (CH₃), 109.5, 117.4, 118.3, 122.2, 123.7 (2×C), 126.2 (2×C), 128.7, 129.3 (2×C), 130.8 (2×C), 130.9, 133.7, 139.8, 141.5, 141.9, 151.4 (Ar—C), 153.8, 162.9 (C═O) p.p.m.

### Example 24

### N-(4-Bromophenyl)-N′-(5-methyl-3-oxo-1,2-diphenyk-2,3-dihydro-1H-pyrazol-4-yl)urea

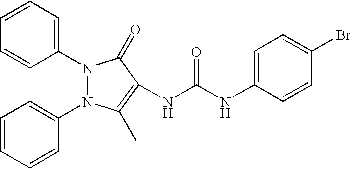

Yield: 92%. Mol. Weight: 463.3. Mol. Formula: C₂₃H₁₉BrN₄O₂. MS (APCI(+)): 464, 466 (M+1), 266 (M+) m/z. IR (KBr-disc) ν max: 3285, 3062, 1704, 1644, 1490, 1534, 1486, 1288, 1209, 757 & 705 cm ⁻¹. ¹H NMR (DMSO-d₆) 300K δ: 2.01 (s, CH₃), 6.50-6.52 (d, Ar—H, J=6.9 Hz), 7.01-7.17 (m, Ar-2H), 7.29-7.43 (m, Ar-11H), 7.65 (s, NH), 9.02 (s, NH) p.p.m. ¹³C NMR(DMSO-d₆) 300K δ: 12.6 (CH₃), 109.6, 113.9, 116.3, 120.7, 123.7 (2×C), 125.9 (2×C), 126.2, 128.7 (2×C), 129.3 (2×C), 130.5, 131.9, 132.0 (2×C), 136.1 (2×C), 139.8, 153.8 (Ar—C), 157.8, 162.9 (C═O) p.p.m.

### Example 25

### N-(5-Methyl-3-oxo-1,2-diphenyl-2,3-dihydro-1H-pyrazol-4yl)N′-2-nitrophenylurea

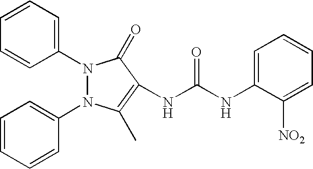

Yield: 65%. Mol. Weight: 430.4. Mol. Formula: C₂₃H₂₀N₅O₄. MS (APCI(+)): 431 (M+1), 266 (M+) m/z. IR (KBr-disc) ν max:3318, 3181, 3010, 1712, 1658, 1635, 1588, 1502, 1432, 1344, 1272, 1201, 759 & 688 cm⁻¹. ¹HNMR (DMSO-d6) 300K δ: 2.02 (s, CH₃), 7.12-7.44 (m, Ar-11H), 7.64-7.71 (t, Ar—H, J=7.3, 7.4 Hz), 8.06-8.10 (d, Ar—H, J=8.4 Hz), 8.28-8.32 (d, Ar—H, J=8.5 Hz), 8.90 (s, NH), 9.71 (s, NH) p.p.m. ¹³C NMR (DMSO-d6) 300K δ: 12.4 (CH₃), 122.7, 123.9, 124.0, 125.9, 126.3 (2×C), 126.6, 127.3 (2×C), 128.8, 128.9, 129.3 (2×C), 130.1 (2×C), 133.2, 135.6, 136.0, 138.0, 139.6 (Ar—C), 153.5, 162.8 (C═O) p.p.m.

### Example 26

### N-(5-Methyl-3oxo-1,2diphenyl-2,3-dihydro-1H-pyrazol4-yl)—N′-phenylurea

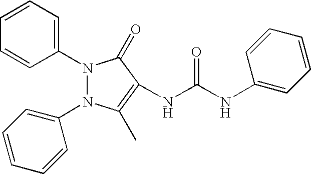

Yield: 91%. Mol. Weight: 384.4. Mol. Formula: C₂₃H₂₀N₄O₄. MS (APCI(+)): 385 (M+1), 266 (M+) ml/z. IR (KBr-disc) ν max: 3420, 3297, 3072, 3065, 1706, 1640, 1544, 1492, 1448, 1297, 1202, 755 & 697 cm-⁻¹. ¹H NMR (DMSO-d₆) 300K δ: 2.02 (s, CH₃), 6.91-6.97 (tt, Ar—H, J=7.3 Hz), 7.11-7.17 (tt, Ar—H, 7.0, 7.1 Hz), 7.22-7.45 (m, Ar-13H), 7.60 (s, NH), 8.87 (s, NH) p.p.m. ¹³C NMR (DMSO-d₆) 300K δ: 12.6 (CH₃), 109.8 (C—CH₃), 118.5 (2×C), 122.2, 122.4 (2×C), 123.7 (2×C), 125.9 (2×C), 126.2, 128.6 129.1 (2×C), 129.8 (2×C), 136.1, 139.9, 140.3, 151.1 (Ar—C), 153.8, 163.0 (C═O) p.p.m. ¹³C NMR (DMSO-d₆) 300K δ: 11.9 (CH₃),36.6 (N—CH₃), 108.9 (CH—N), 118.0, 121.9, 123.4, 124.0, 126.2, 126.3 (2×C), 126.4, 126.5, 126.7 (2×C), 134.2, 134.9 (C—CH₃), 135.2, 135.5, 151.1 (Ar—C), 154.6, 162.6 (C═O) p.p.m.

### Example 27

### N′-(5-Methyl-3-oxo-1,2-diphenyl-2,3dihydro-1H-pyrazol4-yl)urea

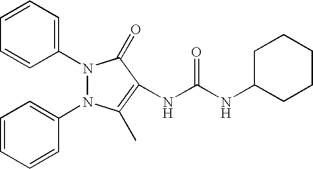

Yield: 86%. Mol. Weight: 390.5. Mol. Formula: C₂₃H₂₃N₄O₂. MS (APCI(+)): 391 (M+1), 266 (M+) m/z. IR (KBr-disc) ν max: 3359; 3299, 2929, 2849, 1636, 1694, 1596, 1538, 1488, 1276, 1228, 763 cm⁻¹. ¹H NMR (DMSO-d₆) 300K δ: 1.10-1.88 (m, —CH, —CH₂13, 11H), 1.95 (s, CH₃), 6.27-6.30 (d, Ar—H, J=7.9 Hz), 7.12-7.16 (tt, Ar—H, J=6.8, 6.9 Hz), 7.24-7.42 (m, Ar-8H), 7.63 (s, NH), 8.86 (NH) p.p.m. ¹³C NMR (DMSO-d₆) 300K 67 : 12.9 (CH₃), 24.9 (—CH₂-×2), 25.8 (—CH₂—), 33.5 (—CH2-×2), 48.5 (—CH—NH), 99.7 (C—CH₃), 110.0 (C—N), 123.5 (2×C), 126.1 (2×C), 128.5, 129.2 (2×C), 130.0 (2×C), 136.1, 140.3, 150.2 (Ar—C), 155.6, 163.1 (C═O) p.p.m.

Examples 28 to 35 were also prepared by analogous methods.

### Examples 36 to 40

By methods analogous to those described above were prepared the following compounds:

### Example 36

### N-(5-Methyl-3-oxo-1,2-diphenyl-2,3dihydro-1H-pyrazol-4yl)-1H-indole-2-carboxamide

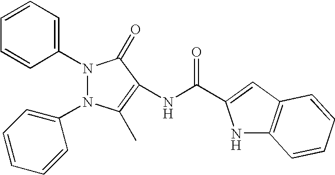

Yield: 65%. Mol. Weight: 408.5. Mol. Formula: C22H₂₀N₄O₂. MS (APCI(−)): 407 (M+1), 364 (M+), 237 (M+) m/z. IR (KBr-disc) ν max: 3401, 3339, 2965, 2358, 1710, 1615, 1583, 1454, 1361, 1172 & 748 cm-⁻¹.

### Example 37

### N-(5-Methyl-3-oxo-1,2-diphenyl-2,3-dihydro-1H-pyrazol-4yl)-1H-indole-3-carboxamide

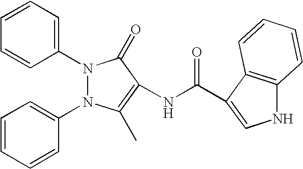

Yield: 78%. Mol. Weight: 408.5. Mol. Formula: C₂₅H₂₀N₄O₂. MS (APCI(+)): 409 (M+1) m/z. IR (KBr-disc) ν max: 3343, 2965, 1615, 1581, 1535, 1494, 1453, 1318, 1249, 1191 & 750 cm⁻¹. ¹H NMR (DMSO-d₆) 300K δ: 2.04 (s, CH₃), 7.09-7.20 (m, Ar—3H), 7.27-7.45 (m, Ar-10H), 7.44-7.47 (d, Ar—H, J=7.0 Hz), 7.99 (s, Ar—H), 9.16 (s, NH), 11.69 (s, NH) p.p.m. ¹³CNMR (DMSO-d₆)300K δ: 12.6(CH₃), 109.7 (C—NH), 112.4, 121.0, 121.1, 121.5, 122.6, 123.6, 126.1(2×C), 126.3, 126.9 (2×C), 128.6 (2×C), 129.2, 129.3 (2×C), 130.1, 132.7, 136.4, 139.9,152.8 (Ar—C), 164.5, 171.9 (C═O) p.p.m.

### Example 38

### N-5-Methyl-3-oxo-1,2-diphenyl-2,3-dihydro-1H-pyrazol4-yl)-2-(1H-indol-3-yl)acetamide

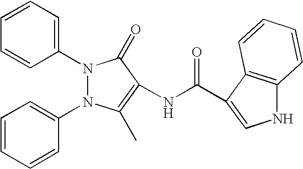

Yield: 66%. Mol. Weight: 422.5. Mol. Formula: C₂₆H₂₂N₄O₂. MS (APCl(+)): 423 (M+1) m/z. IR (KBrdisc) ν max: 3337, 2965, 1679, 1648, 1629, 1592, 1525, 1488, 1312, 1243 & 749 cm⁻¹. ¹H NMR (DMSO-d₆) 300K δ: 1.86 (s, CH₃), 3.73 (s, CH₂), 6.94-7.00 (t, Ar—H, J=8.0, 7.9 Hz), 7.03-7.09 (t, Ar—H, J=8.2, 8.1 Hz), 7.10-7.17 (t, Ar—H, J=6.8, 6.7 Hz), 7.27-7.38 (m, Ar-10H), 7.61-7.64 (d, Ar—H, J=7.7 Hz), 9.38 (s, NH), 10.88 (s, NH) p.p.m. ¹³C NMR (DMSO-d₆) 300K δ: 12.5 (CH₃), 23.8 (CH₂), 109.1 (C—NH), 109.4, 111.8, 118.4, 119.2, 121.5, 123.7 (2×C), 124.4 (2×C), 126.1, 126.4 (2×C), 127.7, 128.6 (2×C), 129.3, 130.0, 136.1, 136.6, 139.7, 151.9 (Ar—C), 162.7, 170.8 (C═O) p.p.m.

### Example 39

### N-(5-Methyl-3-oxo-1,2-diphenyl-2,3-dihydro-1H-pyrazol-4-yl)-3-(1H-indol-3-yl)propanamide

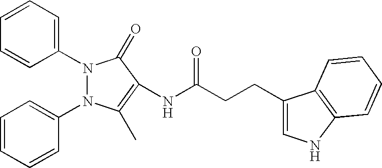

Yield: 79%. Mol. Weight: 436.5. Mol. Formula: C₂₇H₂₄N₄O₂. MS (APCI(+)): 375 (M+1) m/z. IR (KBr-isc) ν max: 3436, 3284, 1640, 1590, 1548, 1490, 1459, 1317 & 753 cm⁻¹. ¹H NMR (DMSO-d₆) 300K δ: 1.84 (s, CH₃), 2.65-2.71 (t, CH₂, J=7.2, 7.1 Hz), 2.98-3.04 (t, CH₂, J=7.3, 7.4 Hz), 6.94-7.00 (t, Ar—H, J=6.8, 6.8 Hz), 7.03-7.09 (t, Ar—H, J=6.9, 6.9 Hz), 7.11-7.17 (m, Ar-2H), 7.27-7.41 (m, Ar-11H), 7.55-7.58 (d, Ar—H, J=7.7 Hz), 9.27 (s, NH), 10.77 (s, NH) p.p.m. ¹³C NMR (DMSO-d₆) 300K δ: 12.5 (CH₃), 21.4, 23.8 (CH₂), 109.3 (C—NH), 111.8, 114.1, 118.6, 118.8, 121.4 (2×C), 122.8, 123.7, 126.1 (2×C), 126.4, 127.6 (2×C), 128.6 (2×C), 129.3, 130.1, 136.2, 136.7, 139.9, 151.9 (Ar—C), 162.7, 172.1 (C═O) p.p.m.

### Example 40

### N-(5Methyl-3-oxo-1,2-diphenyl-2,3-dihydro-1H-pyrazol-4yl)-4-(1H-indol-3-yl)butanamide

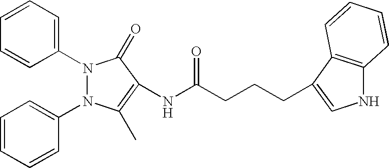

Yield: 80%. Mol. Weight: 450.5. Mol. Formula: C₂₈H₂₆N₄O₂. MS (APCI(+)): 450 (M+1) m/z. IR (KBr-disc) ν max: 3235, 3046, 1656, 1635, 1590, 1544, 1494, 1432, 1276 & 699 cm⁻¹. ¹H NMR (DMSO-d₆) 300K δ: 1.92-1.98 (m, CH₃, CH₂ (overlapping), 2.35-2.40 (t, CH₂, J=7.3, 7.3 Hz), 2.71-2.77 (t, CH₂, J=7.4, 7.5 Hz), 6.93-6.99 (t, Ar—H, J=6.9, 7.2 (m, 7.02-7.10 (t, Ar—H, J=6.9, 6.9 Hz), 7.13-7.16 (t, Ar-2H, J=7.3, 7.1 Hz), 7.24-7.42 (m, Ar-11H), 7.51-7.54 (d, Ar—H, J=7.7 Hz), 9.20 (s, NH), 10.75 (s, NH) p.p.m. ¹³C NMR (DMSO-d₆) 300K δ: 12.5 (CH₃), 23.8,24.8, 26.7 (CH₂), 109.4 (C—NH), 111.8, 114.6,118.4, 118.8, 121.3 (2×C), 122.8, 123.7,126.1 (2×C), 126.4,127.7 (2×C), 128.6 (2×C), 129.5, 130.1, 136.2, 136.8, 139.8, 152.0 (Ar—C), 162.8, 172.4 (C═O) p.p.m.

Pharmacological Methods—^{[125]}I-CCK-8 Receptor Binding Assay

CCK_{A} and CCK_{B} receptor binding assays were performed, by using guinea pig cerebral cortex (CCK_{B}) or rat pancreas (CCK_{A}). Male guinea pig brain tissues were prepared according to the modified method described by Saita et al, [(1994), Characterization of YM022: its CCKB/gastrin receptor binding profile and antagonism to CCK-8-induced Ca2+mobilization., Eur. J. PharmacoL,269, 249-254]. Pancreatic membranes were prepared in a similar way but by Charpentier et al, [(1988), Cyclic cholecystokinin analogues with high selectivity for central receptors., Proc Natl Acad Sci USA, 85, 1968-1972]. For the in vivo CCK binding assay tissues were homogenised in ice cold sucrose (0.32 M, 25 ml) for 15 strokes at 500 rpm and centrifuged at 13000 rpm for 10 mins. The supernatant was re-centrifuged at 13000 rpm for 20 mins. The resulting pellet was re-dispersed to the required volume of buffer at 500 rpm and stored in aliquots at 70° C.

Binding was achieved using a.radioligand ¹²⁵I-Bolton-Hunter labeled CCK, NEN at 25 pM. The samples were incubated {with membranes (0.1 mg/ml)} in 20 mM Hepes, 1 mM EGTA, 5 mM MgCl₂, 150 mm NaCl, 0.25 mg/ml bacitracin at pH 6.5 for 2 hrs at RT and then suspended by centrifugation at 1100 rpm for 5 minutes. The membrane pellets were washed twice with water and the bound radioactivity was measured in a Packard Cobra Auto-gamma counter (B5005). All binding assays were carried out with L-365, 260 (Bock, M. G., et. al., Benzodiazepine gastrin and brain cholecystokinin receptor ligands. L-365,260, J. Med. Chem. 1989, 32, 14-24) as an internal non-specific standard. Controls (no compound) were also added. All samples were made in duplicate and repeated twice. All compounds were initially screened for percentage inhibition at 20 μm. Samples showing an average inhibition of <35% were diluted to 2μm and re-screened and if active diluted again. This enabled the calculation of IC₅₀'s of the most active compounds.

TABLE 1
Biological Evaluation of Phenyl Pyrazolinone Ureas

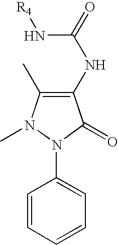

| | Group | Yield | | | MS + H | IC₅₀ CCK_{B} |
|---|---|---|---|---|---|---|
| Example | R₄ | [%] | MW | FW | [m/z] | [μM] |
| 1 | 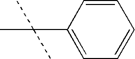 | 94 | 322 | C₁₈H₁₈N₄O₂ | 323 | — |
| 2 | 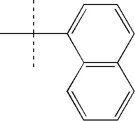 | 91 | 372 | C₂₂H₂₀N₄O₂ | 373 | — |
| 3 | 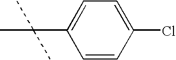 | 75 | 356 | C₁₈H₁₇ClN₄O₂ | 357 | 2.5 |
| 4 | 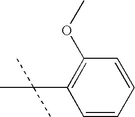 | — | — | — | — | — |
| 5 | 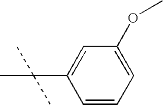 | 93 | 352 | C₁₉H₂₀N₄O₃ | 353 | >10 |
| 6 | 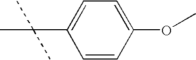 | 97 | 352 | C₁₉H₂₀N₄O₃ | 353 | >10 |
| 7 | 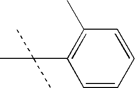 | 90 | 336 | C₁₉H₂₀N₄O₂ | 337 | 7 |
| 8 | 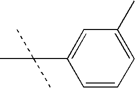 | 95 | 336 | C₁₉H₂₀N₄O₂ | 337 | 5 |
| 9 | 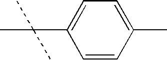 | 92 | 336 | C₁₉H₂₀N₄O₂ | 337 | >20 |
| 10* | 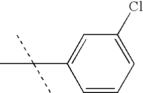 | 89 | 356 | C₁₈H₁₇ClN₄O₂ | 357 | >10 |
| 11 | 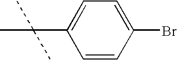 | 82 | 401 | C₁₈H₁₇BrN₄O₂ | 402 | 7 |
| 12 | 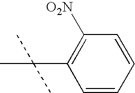 | 84 | 367 | C₁₈H₁₇N₅O₄ | 368 | >20 |
| 13 | 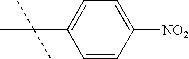 | 94 | 367 | C₁₈H₁₇N₅O₄ | 368 | >20 |
| 14 | 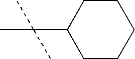 | 85 | 328 | C₁₈H₂₄N₄O₂ | 329 | >10 |
| 15 | 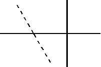 | 78 | 302 | C₁₆H₂₂N₄O₂ | 303 | 8 |

* = Fully characterised

TABLE 2
Biological Evaluation of Phenylpyrazolinone amide Derivatives

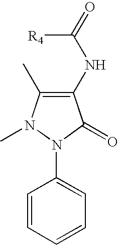

| | | | | | | IC₅₀ | IC₅₀ | |
|---|---|---|---|---|---|---|---|---|
| | Group | Yield | | | MS + H | CCK_{B} | CCK_{A} | Ratio |
| Example | R₄ | [%] | MW | FW | [m/z] | [μM] | [μM] | A/B |
| 16* | 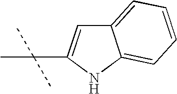 | 76 | 346 | C₂₀H₁₈N₄O₂ | 347 | 0.9 | 0.080 | 11.3 |
| 17* | 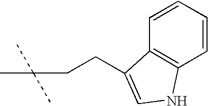 | 75 | 374 | C₂₂H₂₂N₄O₂ | 375 | 4 | 1 | 4 |
| 18 | 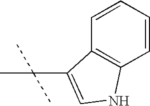 | 80 | 346 | C₂₀H₁₈N₄O₂ | 347 | 15 | 2 | 7.5 |
| 19 | 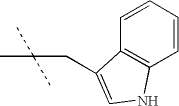 | 82 | 360 | C₂₁H₂₀N₄O₂ | 361 | 9 | 2 | 4.5 |
| 20 | 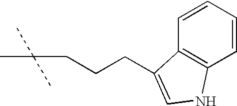 | 78 | 388 | C₂₃H₂₄N₄O₂ | 389 | 20 | 20 | 1 |

* = Fully characterized

TABLE 3
Biological Evaluation of Diphenyl Pyrazolyl Ureas

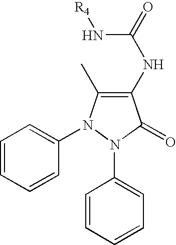

| | | | | | | IC₅₀ | IC₅₀ |
|---|---|---|---|---|---|---|---|
| | Group | Yield | | | MS + H | CCK_{B} | CCK_{A} |
| Example | R₄ | [%] | MW | FW | [m/z] | [μM] | [μM] |
| 21* | 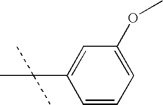 | 67 | 414 | C₂₄H₂₂N₄O₃ | 415 | 0.035 | 0.010 |
| 22* | 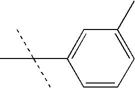 | 91 | 398 | C₂₄H₂₂N₄O₂ | 399 | 0.025 | 0.020 |
| 23* | 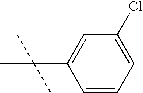 | 73 | 418 | C₂₃H₁₉ClN₄O₂ | 419 | >20 | — |
| 24* | 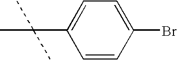 | 92 | 463 | C₂₃H₁₉BrN₄O₂ | 464 | >20 | — |
| 25 | 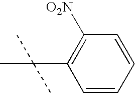 | 65 | 430 | C₂₁H₁₉N₅O₄ | 431 | 7.5 | — |
| 26* | 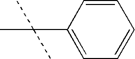 | 91 | 384 | C₂₃H₂₀N₄O₂ | 385 | 3 | — |
| 27* | 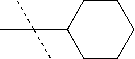 | 86 | 390 | C₂₃H₂₃N₄O₂ | 391 | 0.85 | — |
| 28 | 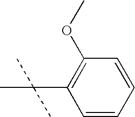 | — | — | — | — | — | — |
| 29 | 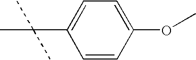 | 57 | 414 | C₂₄H₂₂N₄O₃ | 415 | >20 | — |
| 30 | 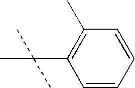 | 88 | 398 | C₂₄H₂₂N₄O₂ | 399 | >20 | — |
| 31 | 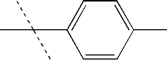 | 89 | 398 | C₂₄H₂₂N₄O₂ | 399 | >20 | — |
| 32 | 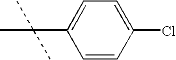 | 81 | 418 | C₂₃H₁₉ClN₄O₂ | 419 | >20 | — |
| 33* | 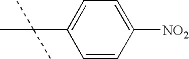 | 77 | 430 | C₂₃H₁₉N₅O₄ | 431 | >20 | — |
| 34 | 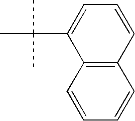 | 75 | 434 | C₂₇H₂₂N₄O₂ | 435 | >20 | — |
| 35 | 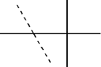 | 60 | 364 | C₂₁H₂₄N₄O₂ | 365 | 1 | — |

TABLE 4
Biological Evaluation of Diphenyl pyrazolinone amide Analogues

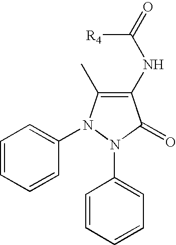

| | | | | | | IC₅₀ | IC₅₀ | |
|---|---|---|---|---|---|---|---|---|
| | Group | Yield | | | MS + H | CCK_{B} | CCK_{A} | Ratio |
| Example | R₄ | [%] | MW | FW | [m/z] | [μM] | [μM] | A/B |
| 36* | 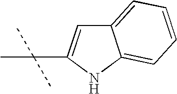 | 65 | 408 | C₂₅H₂₀N₄O₂ | 409 | 0.030 | 0.020 | 1.5 |
| 37* | 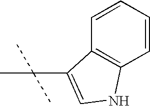 | 78 | 408 | C₂₅H₂₀N₄O₂ | 409 | 3.5 | 2 | 1.8 |
| 38* | 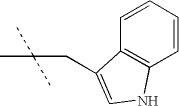 | 66 | 422 | C₂₆H₂₂N₄O₂ | 423 | 2.5 | 0.020 | 125 |
| 39* | 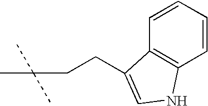 | 79 | 436 | C₂₇H₂₄N₄O₂ | 437 | 2 | 0.025 | 80 |
| 40* | 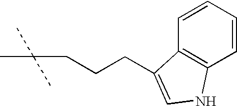 | 80 | 450 | C₂₈H₂₆N₄O₂ | 451 | 20 | 20 | 1 |

* = Full charcaterised

What is claimed is:

1. A compound of formula (I): 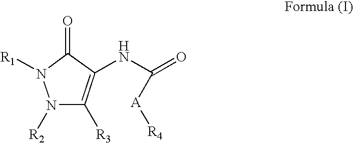
wherein each of R₁ to R₄ is independently selected from hydrogen, a halogen, a substituted or unsubstituted cyclic and heterocyclic moiety, substituted or unsubstituted, linear or branched alkyl, alkyloxy, alkylcarbonyl, alkyloxycarbonyl, alkenyl,alkenyloxy, alkenylcarbonyl, alkenyloxycarbonyl, alkynyl, alkynyloxy, alkynylcarbonyl, alkynyloxycarbonyl, aryl, benzyl, aryloxy, arylcarbonyl, aryloxycarbonyl and sulphur equivalents of said oxy, carbonyl and oxycarbonyl moieties, and A is NH, or (CH₂)ₙ, where n is 0,1 or 2, and physiologically acceptable salts and hydrates thereof.

2. A compound as claimed in claim 1, wherein said alkyl-containing moieties are C₁-C₁₂.

3. A compound as claimed in claim 1, wherein said alkenyl- and said alkynyl-containing moieties are C₂-C₁₂.

4. A compound as claimed in claim 1, wherein said aryl moiety is substituted or unsubstituted phenyl, napthyl or indolyl.

5. A compound as claimed in claim 4, wherein said aryl moiety is m-substituted phenyl, indol-2yl and or-3-yl.

6. A compound as claimed in claim 1, wherein said substituents for said heterocyclic, alkyl, alkenyl, alkynyl and aryl moieties are independently selected from halo, amino, nitro, hydroxy, alkoxy and cyano moieties.

7. A compound as claimed in claim 1, wherein said heterocyclic moiety is a monocyclic or bicyclic ring comprising at least one of oxygen, sulphur and nitrogen.

8. A compound as claimed in claim 1, wherein said cyclic alkyl moiety is a 3 to 7 membered ring and said cyclic alkenyl and alkynyl moieties are 4 to 7 membered rings.

9. A compound as claimed in claim 1, wherein R₁ is selected from H, C₁₋₄ alkyl, phenyl, benzyl, cyclohexyl, and a heterocyclic moiety.

10. A compound as claimed in claim 9, wherein R₁ is phenyl.

11. A compound as claimed in claim , wherein R₂ is selected from H, C₁₋₄ alkyl, phenyl, aryl, CH₂-heterocyclic moiety, CH₂CO-alkyl, CH₂CO-aryl, benzyl, cyclohexyl, and cycloalkyl.

12. A compound as claimed in claim 11, wherein R₂ is phenyl or methyl.

13. A compound as claimed in claim 1, wherein R₃ is selected from H, methyl, alkyloxy, aryloxy and a halogen.

14. A compound as claimed in claim 13, wherein R₃ is methyl.

15. A compound as claimed in claim 1, wherein R₄ is selected from aryl, a cyclic alkyl moiety or a heterocyclic moiety.

16. A compound as claimed in claim 15, wherein R₄ is selected from indolyl and cyclohexyl.

17. A compound as claimed in claim 1, wherein R₄ is mono-substituted phenyl, t-butyl, cyclohexyl or indol-2-yl when A is NH.

18. A compound as claimed in claim 1, wherein R₄ is indol-2-yl or indol-3-yl when A is(CH₂)ₙ.

19. A compound as claimed in claim 1 having any of the formulae (II) to (XV):

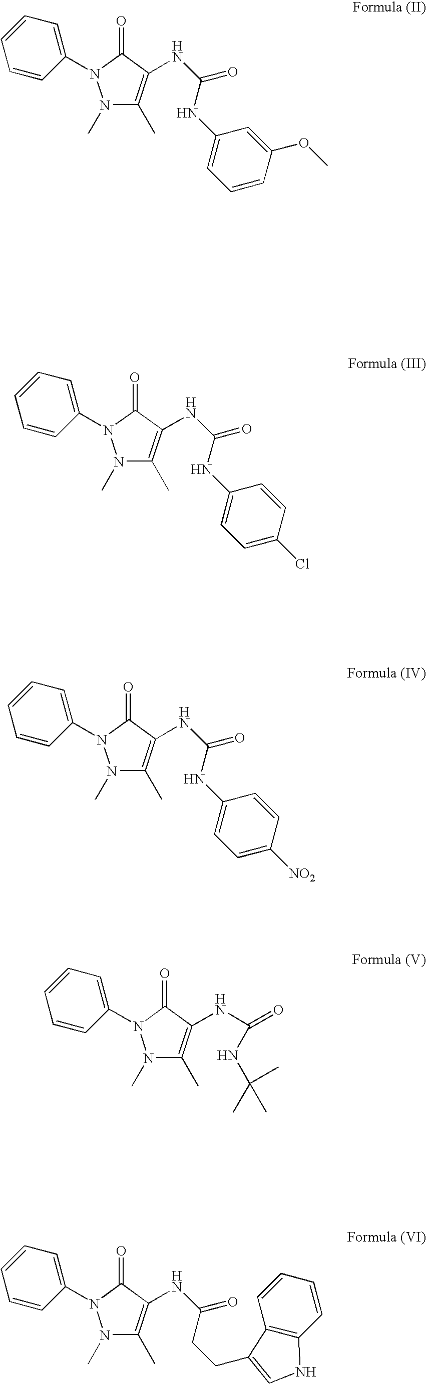

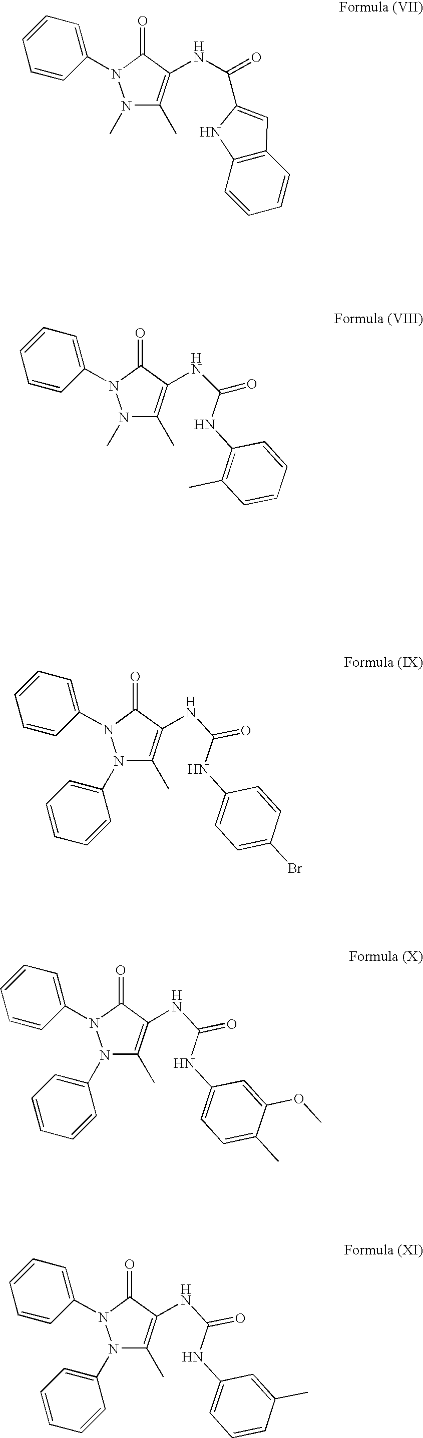

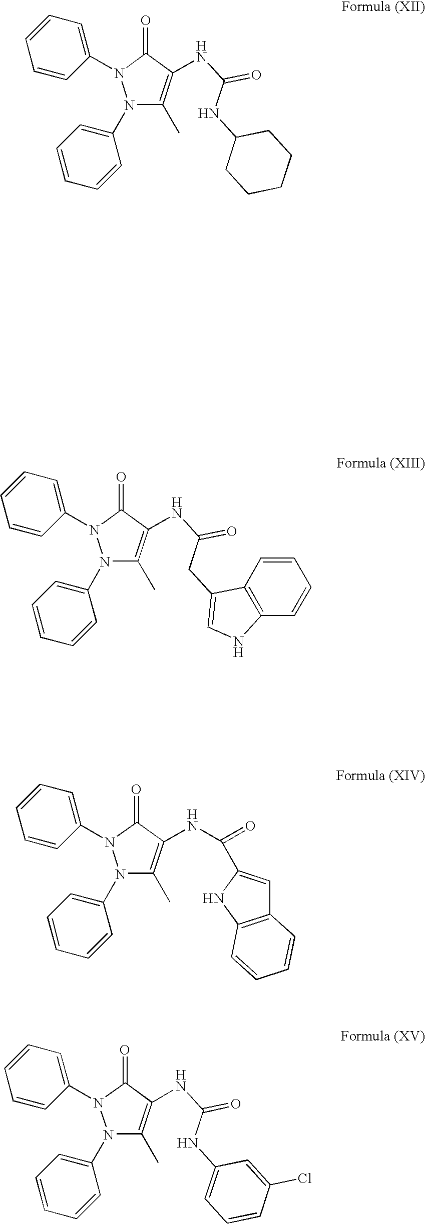

20. A method of producing a compound of formula (I), comprising the sequential steps of:
(i) reacting a di-substituted hydrazine derivative of formula (1) with a β-ketoester of formula (2) to produce a pyrazolone of formula (3),
(ii) introducing an amine group at the 4-position of the pyrazolone (3) to produce 4-aminopyrazolone (4), and
(iii) reacting the aminopyrazolone (4) with either an isocyanate of formula (5) to produce the desired ureido-pyrazolone (6), or a carboxylic acid of formula (7) to produce the desired amido-pyrazolone (8), 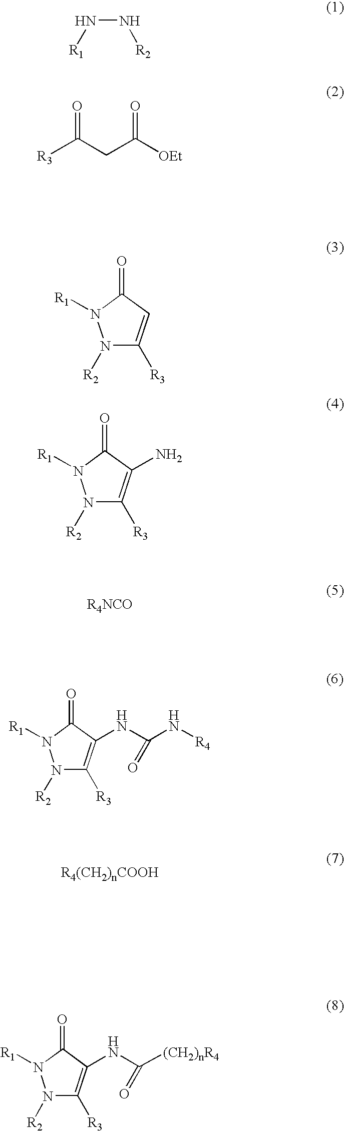
R₁ to R₄ and n being as defined in claim 1.

21. The method of claim 20 including the additional step of alkylating the pyrazolone (3) prior to step (ii) when R₂ is H.

22. The method of claim 20, wherein the di-substituted hydrazine used in step (i) is a phenylhydrazine (R₁ is Ph).

23. The method of claim 20, wherein step (ii) is achieved by introducing a nitroso group at the 4-position of the pyrazolone (3) followed by reduction.

24. (canceled)

25. (canceled)

26. A method of treatment of a mammal afflicted with a CCK-receptor mediated condition, or prophylaxis in a mammal at risk of a CCK-receptor mediated condition comprising administering a therapeutically effective amount of a compound as claimed in claim 1.

27. (canceled)

28. The method of claim 26, wherein said CCK-receptor mediated conditions is a GI disorder, a CNS disorder caused by CCK interaction with dopamine, another CNS disorder; oncologic disorder, disorder of appetite regulatory systems; Zollinger-Ellison syndrome; antral G cell hyperplasia; or pain.

29. The method of claim 28, wherein said GI disorder is selected from irritable bowel syndrome, gastro-oesophageal reflux disease or ulcers, excess pancreatic or gastric secretion, acute pancreitis,or motility disorders; said CNS disorder is selected from neuroleptic disorders, tardive dyskinesia, Parkinson's disease, psychosis or Gilles de la Tourette syndrome, said another CNS disorder is selected from anxiety disorders and panic disorders and said oncologic disorder is selected from small cell adenocarcinomas and primary tumours of the central nervous system glial and neuronal cells.

30. A method of inhibiting CCK receptor activity comprising contacting a composition comprising a CCK receptor with the compound of claim 1.

31. The method of claim 30, wherein said ligand is a selective CCK1 or CCK2 ligand.

32. A composition for the treatment or prophylaxis of a CCK-receptor mediated condition comprising the compound of claim 1 and a pharmaceutically acceptable carrier.
